(19) United States
(12) Patent Application Publication
(10) Pub. No.: US 2019/0060337 A1
(43) Pub. Date: Feb. 28, 2019

(54) COMPOSITIONS COMPRISING BETA-GLUCOGALLIN AND THERAPEUTIC APPLICATIONS THEREOF IN CONTROLLED KINETICS OF CARBOHYDRATE BREAKDOWN AND MONOSACCHARIDE ABSORPTION

(71) Applicants: Muhammed Majeed, Edison, NJ (US); Kalyanam Nagabhushanam, East Windsor, NJ (US); Sivakumar Arumugam, Bangalore (IN); Furqan Ali, Bangalore (IN)

(72) Inventors: Muhammed Majeed, Edison, NJ (US); Kalyanam Nagabhushanam, East Windsor, NJ (US); Sivakumar Arumugam, Bangalore (IN); Furqan Ali, Bangalore (IN)

(21) Appl. No.: 15/971,069

(22) Filed: May 4, 2018

(30) Foreign Application Priority Data
Aug. 31, 2017 (IN) 201741030866

(51) Int. Cl. A61K 31/7032; A61P 3/10

(52) U.S. Cl. CPC A61K 31/7032; A61K 31/235; A61K 2300/00; A61P 3/10

(57) ABSTRACT

Disclosed are the compositions for the effective regulation of carbohydrate breakdown and absorption. More specifically, the invention discloses compositions containing at least 10% w/w or above of 1-O-galloyl-β-D-glucose (β-glucogallin) and additionally comprising of about 10% w/w to greater than 60% w/w total mucic acid gallates for the effective regulation of carbohydrate breakdown and absorption by the inhibition of enzymes amylase, glucosidase and dipeptidyl peptidase.

### CROSS REFERENCE TO RELATED APPLICATION

This is a conventional US patent application claiming priority from Indian Provisional application no. 201741030866 filed on 31 Aug. 2017, the details of which are being incorporated herein by reference.

### BACKGROUND OF THE INVENTION

### Field of the Invention

The present invention relates to therapeutic intervention for regulating carbohydrate breakdown and absorption in mammals. More specifically, the present invention relates to compositions comprising β-glucogallin and therapeutic applications thereof in controlled kinetics of carbohydrate breakdown and monosaccharide absorption.

### Description of Prior Art

Hyperglycemia is characterized by presence of elevated levels of glucose in blood. If left untreated, the condition may lead to many diseases like diabetes, obesity, hyperlipoproteinemia, hyperlipidemia, cardiovascular complications, cancer, atherosclerosis, allergy, inflammation, and osteoporosis. Currently there are many drugs which are administered to control the blood glucose levels (e.g metformin). But increased intake of these drugs increases stress on the kidneys leading to damage and nephrotoxicity. Thus, current research is targeted at developing a natural plant based drug which is safe, cheap and effective in managing the blood glucose levels. While there are many treatment methods available to reduce the aberrant glucose levels, developing inhibitors against key enzymes that regulate the carbohydrate breakdown and absorption can be a potential target, as these enzymes catalyze the most important biochemical pathways. Controlled kinetics of carbohydrate digestion and monosaccharide absorption could be of great value in the avoidance of aforementioned disease conditions. In this aspect, amylase, glucosidase and dipeptidyl peptidase inhibitors are of particular importance (Kim et al. (2004) Isolation and characterization of α-glucosidase inhibitor from the fungus Ganoderma lucidum. Journal of Microbiology, 42, 223-227; Ahren et al. (2004) Inhibition of Dipeptidyl peptidase-4 Reduces Glycemia, Sustains Insulin Levels, and Reduces Glucagon Levels in Type 2 Diabetes, The Journal of Clinical Endocrinology & Metabolism, 89 (5), 2078-2084).

α-Amylase (E.C.3.2.1.1) is a hydrolase enzyme catalysing the hydrolysis of α-1, 4-glycosidic linkages in carbohydrates (starch) to yield monosaccharide units like glucose and maltose. α-glucosidase, is essential for the degradation of glycogen to glucose. Both these enzymes act on complex carbohydrate molecules to yield monosaccharide units which are readily absorbed in the blood stream. Dipeptidyl peptidase-4 is another key enzyme which increases the blood glucose level by inhibiting the action of Incretins (metabolic hormones that stimulate a decrease in blood glucose levels by promoting insulin secretion) Inhibiting the activities of the above key enzymes would help in regulating the breakdown and uptake of carbohydrates, thereby decreasing the hyperglycemic condition.

There are many plant based inhibitory molecules of these enzymes which are discussed in the following prior arts:
1. Alagesan et al, “Amylase inhibitors: Potential source of anti-diabetic drug discovery from medicinal plants”, Int. J. of Pharm. & Life Sci. (IJPLS), Vol. 3, Issue 2: February: 2012, 1407-1412.
2. Da Silva et al., “Inhibition of salivary and pancreatic α-amylases by a pinhão coat (Araucaria angustifolia) extract rich in condensed tannin” Food Research International. Volume 56, February 2014, Pages 1-8
3. Borde et al., “Dipeptidyl peptidase-iv inhibitory activities of medicinal plants: Terminalia arjuna, Commiphora mukul, Gymnema sylvestre, Morinda citrifolia, Emblica officinalis” Asian J Pharm Gin Res, Vol 9, Issue 3, 2016, 180-182
4. Thilagam et al., “α-Glucosidase and α-Amylase Inhibitory Activity of Senna surattensis” Journal of Acupuncture and Meridian Studies, Volume 6, Issue 1, February 2013, Pages 24-30
5. Poongunran et al., “α-Glucosidase and α-Amylase Inhibitory Activities of Nine Sri Lankan Antidiabetic Plants”, British Journal of Pharmaceutical Research, 7(5): 365-374, 2015.

However, a plant based molecule that effectively inhibits key enzymes in carbohydrate metabolism, especially, amylase, glucosidase and dipeptidyl peptidase is lacking and there exists an unmet industrial need to find an active plant based molecule that is cheap, non-toxic and effective. The present invention solves the abovementioned problem by disclosing compositions comprising β-glucogallin for the management of aberrant carbohydrate metabolism in mammals.

It is the principle objective of the invention to disclose compositions containing β-glucogallin the maintenance of carbohydrate homeostasis in mammals by the inhibition of enzymes amylase, glucosidase and dipeptidyl peptidase

The present invention fulfils aforesaid objectives and provides further related advantages.

### SUMMARY OF THE INVENTION

The present invention discloses compositions for the effective regulation of carbohydrate breakdown and absorption. More specifically, the invention discloses compositions containing at least 10% w/w or above of 1-O-galloyl-β-D-glucose (β-glucogallin) and additionally comprising of about 10% w/w to greater than 60% w/w total mucic acid gallates including mucic acid 1,4-lactone 5-O-gallate, mucic acid 2-O-gallate, mucic acid 6-Methyl ester 2-O-gallate, mucic acid 1-Methyl ester 2-O-gallate and ellagic acid for the effective regulation of carbohydrate breakdown and absorption by the inhibition of enzymes amylase, glucosidase and dipeptidyl peptidase.

Other features and advantages of the present invention will become apparent from the following more detailed description, taken in conjunction with the accompanying drawings, which illustrate, by way of example, the principle of the invention.

### BRIEF DESCRIPTION OF DRAWINGS

FIG. 1 shows the graphical representation of pancreatic α amylase inhibition activity of the compositions comprising at least 10% w/w of 1-O-galloyl-β-D-glucose (β-glucogallin) and 10% w/w to 60% w/w mucic acid gallates.

FIG. 2 shows the graphical representation of human salivary α amylase inhibition activity of the compositions comprising at least 10% w/w of 1-O-galloyl-β-D-glucose (β-glucogallin) and 10% w/w to 60% w/w mucic acid gallates.

FIG. 3 shows the graphical representation of a glucosidase inhibition activity of the compositions comprising at least 10% w/w of 1-O-galloyl-β-D-glucose (β-glucogallin) and 10% w/w to 60% w/w mucic acid gallates.

FIG. 4 shows the graphical representation of Dipeptidyl peptidase-4 Inhibition activity of the compositions comprising at least 10% w/w of 1-O-galloyl-β-D-glucose (β-glucogallin) and 10% w/w to 60% w/w mucic acid gallates.

### DESCRIPTION OF THE MOST PREFERRED EMBODIMENTS

In a most preferred embodiment, the invention discloses a method of inhibiting key enzymes involved in carbohydrate metabolism, said method comprising steps of:
i) Bringing into contact one of the key enzymes in carbohydrate metabolism with a suitable substrate;
ii) Incubating with an effective concentration of a composition containing at least 10% w/w of 1-O-galloyl-β-D-glucose (β-glucogallin) and 10% w/w to 60% w/w mucic acid gallates under optimal conditions;
iii) Reading the change in absorbance using spectrophotometric and fluorimetric methods
iv) Comparing the absorbance with a control blank and determining the percentage enzyme inhibition (IC₅₀) by the said composition containing at least 10% w/w of 1-O-galloyl-β-D-glucose (β-glucogallin) and 10% w/w to 60% w/w mucic acid gallates using the formula:

% Inhibition=[(absorbance of control−absorbance of inhibitor)/absorbance of control]×100.

In a related embodiment, the mucic acid gallates are selected from the group consisting of mucic acid 1,4-lactone 5-O-gallate, mucic acid 2-O-gallate, mucic acid 6-Methyl ester 2-O-gallate, mucic acid 1-Methyl ester 2-O-gallate and ellagic acid. In another related embodiment, the enzymes are selected from the list consisting of pancreatic α amylase, salivary α amylase, α glucosidase and dipeptidyl peptidase-4.

In another most preferred embodiment, the invention discloses a composition containing at least 10% w/w of 1-O-galloyl-β-D-glucose (β-glucogallin) and 10% w/w to 60% w/w mucic acid gallates for the management of hyperglycemic conditions in mammals by inhibiting carbohydrate absorption, and normalizing the metabolism of glucose. In a related embodiment, the mucic acid gallates are selected from the group consisting of mucic acid 1,4-lactone 5-O-gallate, mucic acid 2-O-gallate, mucic acid 6-Methyl ester 2-O-gallate, mucic acid 1-Methyl ester 2-O-gallate and ellagic acid. In another related embodiment, the effective regulation of carbohydrate absorption and breakdown reduces hyperglycemic conditions present in disease states selected from the group consisting of diabetes, obesity, hyperlipoproteinemia, hyperlipidemia, cardiovascular complications, cancer, atherosclerosis, allergy, inflammation, and osteoporosis. In another related embodiment, the mammal is human. In another related embodiment, the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.

In another preferred embodiment, the invention discloses a method for the therapeutic management of hyperglycemia in mammals by inhibiting the activity of key enzymes, said method comprising steps of administering a composition containing at least 10% w/w of 1-O-galloyl-β-D-glucose (β-glucogallin) and 10% w/w to 60% w/w mucic acid gallates, to bring about the inhibition of key enzymes involved in carbohydrate metabolism. In a related embodiment, the inhibition of enzymes results in decreasing blood glucose levels by regulating the release of glucose into the blood and cellular uptake of glucose from the blood stream. In a related embodiment, the mucic acid gallates are selected from the group consisting of mucic acid 1,4-lactone 5-O-gallate, mucic acid 2-O-gallate, mucic acid 6-Methyl ester 2-O-gallate, mucic acid 1-Methyl ester 2-O-gallate and ellagic acid. In another related embodiment, the enzymes are selected from the list consisting of pancreatic α-amylase, salivary α-amylase, α-glucosidase and dipeptidylpeptidase-4. In another related embodiment, the hyperglycemia condition is present in disease states selected from the group consisting of diabetes, obesity, hyperlipoproteiniemia, hyperlipidemia, cardiovascular complications, cancer, atherosclerosis, allergy, inflammation, and osteoporosis. In another related embodiment, the mammal is human. In another related embodiment, the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.

In another preferred embodiment, the invention discloses a method for the therapeutic management of hyperglycemia by increasing secretion of insulin in mammals, using a composition containing at least 10% w/w of 1-O-galloyl-β-D-glucose (β-glucogallin) and 10% w/w to 60% w/w mucic acid gallates to said mammals, to bring about the inhibition of enzyme dipeptidyl peptidase-4 thereby promoting insulin secretion and increased cellular uptake of glucose from the blood. In a related embodiment the mucic acid gallates are selected from the group consisting of mucic acid 1,4-lactone 5-O-gallate, mucic acid 2-O-gallate, mucic acid 6-Methyl ester 2-O-gallate, mucic acid 1-Methyl ester 2-O-gallate and ellagic acid. In another related embodiment the said composition is used for the management of hyperglycemia present in disease conditions selected from the group consisting of diabetes, obesity, hyperlipoproteinemia, hyperlipidemia, cardiovascular complications, cancer, atherosclerosis, allergy, inflammation, and osteoporosis. In a preferred embodiment, the mammal is human. In another preferred embodiment, the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.

The aforesaid most preferred embodiments incorporating the technical features and technical effects of instant invention, are explained through illustrative examples herein under.

### Example 1: Inhibition of α Amylase

One unit of enzyme pancreatic α-amylase (Sigma-Aldrich, St. Louis, Mo., USA) or salivary α-amylase (Sigma-Aldrich) was prepared using 0.1M sodium acetate buffer of pH 4.8. One ml of enzyme was mixed with one ml of 500, 250, 125, 62.5, 31.3, 15.6 and 7.8 μg/ml of compositions comprising at least 10% w/w of 1-O-galloyl-β-D-glucose (β-glucogallin) and 10% w/w to 60% w/w mucic acid gallates including mucic acid 1,4-lactone 5-O-gallate, mucic acid 2-O-gallate, mucic acid 6-Methyl ester 2-O-gallate, mucic acid 1-Methyl ester 2-O-gallate and ellagic acid was used as test and Acarbose as internal experimental control, mixed and kept in water bath at 25° C. for 10 min. After 10 min one ml of reaction mixer was mixed with 1 ml of 0.5% starch and further incubated at 25° C. for 30 min. To stop the reaction 1 ml of Dinitrosalicylic acid reagent was added mixed and placed in boiling water bath for 15 min, cooled to room temperature. After the boiling, 9 ml of DM water was added and cooled to room temperature. The absorbance was recorded at 540 nm.

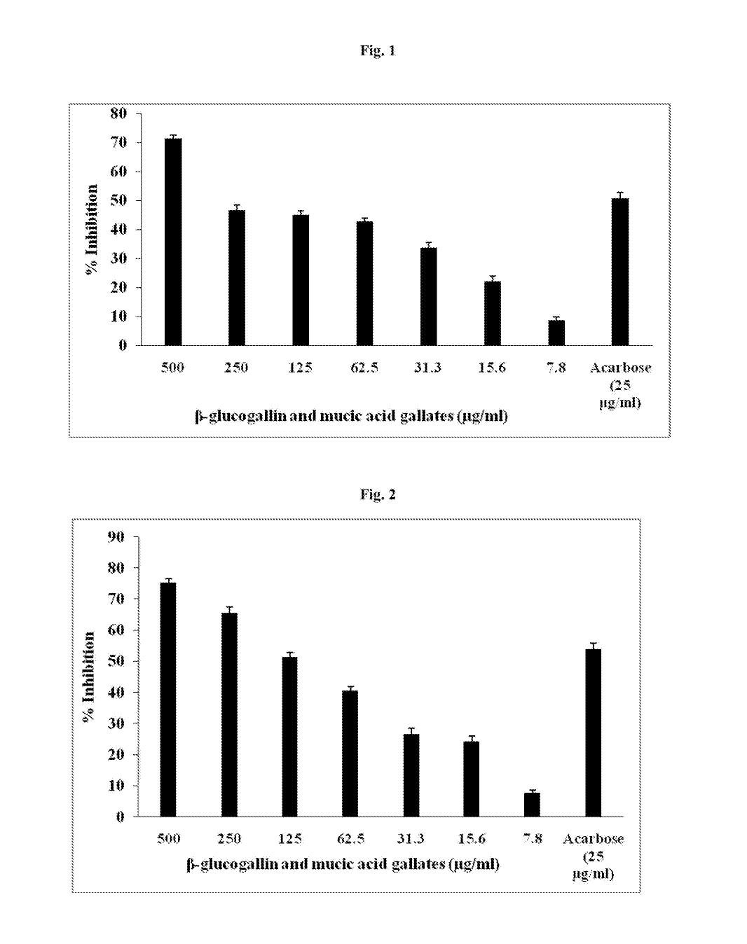

The composition containing β glucogallin and 10% w/w to 60% w/w mucic acid gallates exhibited significant inhibition of pancreatic α amylase and salivary α amylase. Seven different concentration were tested, the said composition showed good inhibitory effect at all the tested concentrations (500, 250, 125, 62.5, 31.3 15.6 and 7.8 μg/ml) but at a higher concentration of 500 μg/ml the maximum inhibitory effect of said composition was showed significant pancreatic α amylase and salivary α-amylase inhibitory activity (72.16%, 75.24%) with IC₅₀ value of 252.6 μg/ml, 180.62 μg/ml respectively (FIG. 1 and FIG. 2). The experiment was repeated for three times and results expressed as mean value of inhibition activity percentage.

### Example 2: Inhibition of a Glucosidase

For a glucosidase inhibition, α-glucosidase (Code G5003; Sigma-Aldrich, St. Louis, Mo., USA) was dissolved in 67 mM potassium phosphate buffer, pH 6.8, containing 8 containing 0.2% Bovine Serum Albumin (Sigma-Aldrich) & 0.02% sodium azide (Sigma-Aldrich) which was used as enzyme source. Paranitrophenyl-α-d-glucopyranoside (Sigma-Aldrich) was used as substrate. Compositions comprising at least 10% w/w of 1-O-galloyl-β-D-glucose (β-glucogallin) and 50% mucic acid gallates including mucic acid 1,4-lactone 5-O-gallate, mucic acid 2-O-gallate, mucic acid 6-Methyl ester 2-O-gallate, mucic acid 1-Methyl ester 2-O-gallate and ellagic acid was weighed and serial dilutions of 62.5, 125, 250, 500, 1000 μg/ml were made up with equal volumes of distilled water. 50 μl of said composition was incubated for 5 min with 50μl enzyme source (0.15 U/ml). After incubation, 50 μl of substrate (1.25 mM) was added and further incubated for 20 min at room temperature. Presubstrate and post-substrate addition, absorbance was measured at 405 nm on a microplate reader (BMG FLUOstar OPTIMA Microplate Reader). The increase in absorbance on substrate addition was obtained. Each test was performed three times and the mean absorption was used to calculate percentage α-glucosidase inhibition. Acarbose was used as positive control with various concentrations. The inhibitory activities of varying concentrations of said composition were expressed as 100 minus the absorbance difference (%) of the said composition relative to the absorbance change of the negative control (i.e., water used as the test solution). The measurements were performed in triplicate, and the IC₅₀ value (i.e., the concentration of said composition that result in 50% inhibition of maximal activity) was determined.

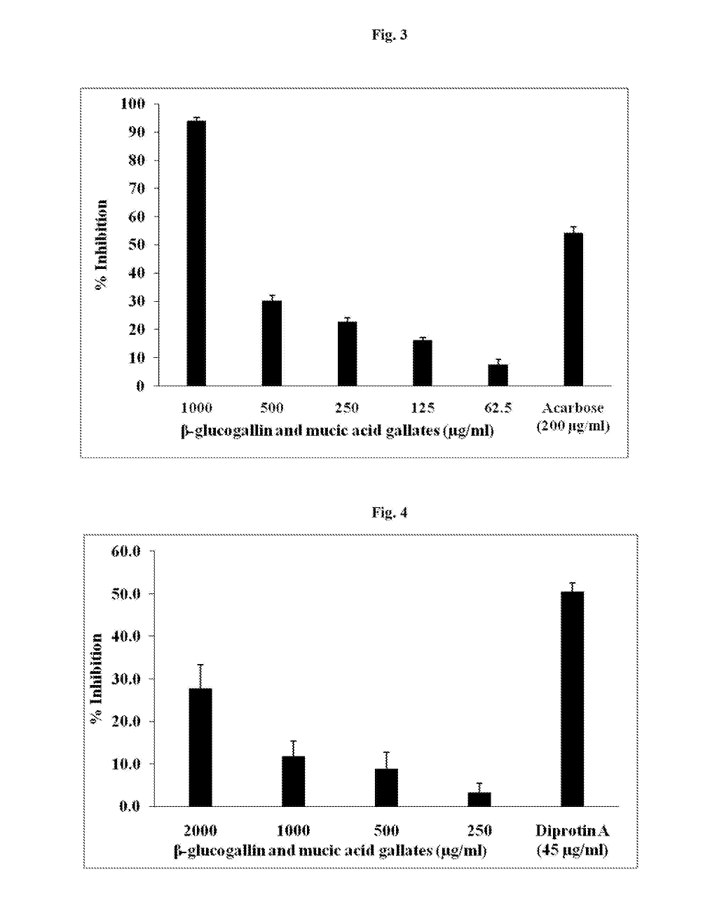

The composition exhibited concentration dependant inhibition of a glucosidase with maximum inhibition (93.92%) at a concentration of 1000 μg/ml (FIG. 3).

### Example 3: Inhibition of Dipeptidyl Peptidase-4

To determine the ability of the compositions comprising at least 10% w/w of 1-O-galloyl-β-D-glucose (β-glucogallin) and 10% w/w to 60% w/w mucic acid gallates including mucic acid 1,4-lactone 5-O-gallate, mucic acid 2-O-gallate, mucic acid 6-Methyl ester 2-O-gallate, mucic acid 1-Methyl ester 2-O-gallate and ellagic acid to inhibit dipeptidyl peptidase-4 (DPP-IV) enzyme, biochemical assay was performed. The assay was performed in 96-well plate. Recombinant Human DPPIV/CD26 (rhDPP1V) (Sigma-Aldrich) was used as the enzyme source. 25 nM Tris HCL, pH 8 was used as assay buffer. Gly-Pro-7-amido-4-methylcoumarin hydrobromide (H-Gly-Pro-AMC; Bachem, Catalog: 1-1225)) was used as the substrate. Said composition was dissolved in water and concentrations ranging from 250 to 2000 μg/ml were taken for the assay. 50 μl, of 0.2 ng/μL of rhDPPIV was added to the plate and start the reaction by adding 50 μL of 20 μM Substrate. As a Substrate Blank combine 50 μL of Assay Buffer and 50 μL of Substrate. The assay was run by mixing 70 μl of 0.1 M Tris buffer, 50 μL of 0.2 ng/μL of rhDPPIV and 30 μl of the test sample. After a 15 minute incubation period at 37° C., 50 μL of substrate solution (20 μM) was added to the appropriate wells to commence the reaction. Following an incubation period of 30 minutes at 37° C., fluorescence was determined at excitation and emission wavelengths of 380 nm and 460 nm, respectively, in kinetic mode for 5 minutes. The assay was performed in triplicate with appropriate blanks and standard. Diprotin A was used as internal experimental control for the study. The percentage of inhibition was calculated using the following formula: % Inhibition=[(absorbance of control−absorbance of inhibitor)/absorbance of control]×100.

The said composition inhibited the activity of an enzyme Dipeptidyl peptidase-4 (DPP-4) in a concentration dependant manner with maximum inhibition (27.9%) at a concentration of 2000 μg/ml (FIG. 4).

### Example 4: Compositions/Formulations Containing β-Glucogallin

Given that β-glucogallin is useful in regulating blood glucose levels and its non toxic nature (LD₅₀ approx. 2000 mg/kg bodyweight), it can be effectively blended in different compositions/formulations that has potential role in therapeutic indications wherein carbohydrate homeostasis play a vital role viz. maintenance and rejuvenation of general health, blood glucose support, for cardiac health, as an antioxidant, for regulating digestion, energy and sports endurance.

Tables 1-6 provide illustrative examples of food formulations containing β-glucogallin and mucic acid gallates for regulating carbohydrate homeostasis

TABLE 1
Rejuvenation blend (Premix) containing β-glucogallin

Active Ingredients
β-glucogallin and mucic acid gallates (50-500 mg), Taurine
Excipients
Fructose, Citric Acid, Tartaric Acid, Sucralose, Lemon flavour and
artificial Peppermint Flavour

Directions for use: Dissolve the required premix in 200-300 ml water and mix well before use

TABLE 2
Digestive support blend (Premix) containing β-glucogallin

Active Ingredients
β-glucogallin and mucic acid gallates (50-500 mg), Bacillus coagulans
MTCC 5856, Fenumannans, Triphala Aquasol, 20% Gingerols,
Excipients
Maltodextrin, Citric Acid, Malic Acid, Sucralose, Lime, Spearmint and
Mangoginger flavours and artificial Mint Flavour, Cumin powder, Black
Salt powder, Asafoetida
Directions for use: Dissolve the required premix in 200-300 ml water and mix well before use

TABLE 3
Blood glucose support blend (Premix) containing β-glucogallin

Active Ingredients
β-glucogallin and mucic acid gallates (100-1000 mg), Fenumannans,
Moringa leaf extract, Pterocarpus water soluble extract, Aloe Powder,
Vitamin B6, Vitamin B12
Excipients
Maltodextrin, Taurine, Citric Acid, Malic Acid, Sucralose, Trisodium
Citrate, Contains Nature identical Apple flavour and artificial Peppermint
Flavour and Bitterness masker flavor
Directions for use: Dissolve the required premix in 200-300 ml water and mix well before use

TABLE 4
Energy blend (Premix) containing β-glucogallin

Active Ingredients
β-glucogallin and mucic acid gallates (100-1000 mg), Taurine, Inositol,
L-Arginine, Cococin, GlucoronoLactone, L-Selenomethionine, Zinc
Monomethionine, Magnesium Gluconate, Calcium Citrate Malate,
Ascorbic Acid, B Vitamin Blend - Vitamin B3, Vitamin B6, Vitamin B9,
Vitamin B12
Excipients
Carageenan, Citric Acid, Tartaric Acid, Trisodium Citrate, Sucralose,
Lime flavour and artificial Pepeprmint Flavour, Synthetic food colour.
Directions for use: Dissolve the required premix in 200-300 ml water and mix well before use

TABLE 5
Sports support blend (Premix) containing β-glucogallin

Active Ingredients
β-glucogallin and mucic acid gallates (200-2000 mg), Taurine, SaBeet ®
(beet root extract), L-Arginine, Grapeseed Extract, Moringa leaf extract,
B Vitamins - Vitamin B6, Vitamin B12
Excipients
Maltodextrin, Xylitol, Citric Acid, Malic Acid, Potassium Chloride,
Sucralose, Strawberry flavor, and also contains permitted Class II
presrvative (Sodium Benzoate).
Directions for use: Dissolve the required premix in 200-300 ml water and mix well before use
® Registered TM of Sabinsa Corporation, USA

TABLE 6
Anti-oxidant/Cardiac health blend (Premix) containing β-glucogallin

Active Ingredients
β-glucogallin and mucic acid gallates (50-500 mg), Beetroot Extract,
Citrin Crystals (Anthocyanins), Moringa Leaf Extract, B Vitamins -
Vitamin B6 and Vitamin B12, Fruit Powder (Orange, Banana, Pineapple,
Apple, Pomegranate, Jamun, Cranberry, Grape)
Excipients
Maltodextrin, Xylitol, Citric Acid, Malic Acid, Potassium Chloride,
Sucralose, Calcium Silicate, Strawberry flavour.
Directions for use: Dissolve the required premix in 200-300 ml water and mix well before use

Tables 7 and 8 provides illustrative examples of nutraceutical formulations containing β-glucogallin and mucic acid gallates for regulating carbohydrate homeostasis

TABLE 7
β-glucogallin Tablet

Active Ingredients
β-glucogallin and mucic acid gallates (50-500 mg)
Excipients
Microcrystalline cellulose, Hypromellose, Croscarmellose Sodium,
Colloidal silicon dioxide, Magnesium stearate

TABLE 8
β-glucogallin Capsule

Active Ingredients
β-glucogallin and mucic acid gallates (50-500 mg)
Excipients
Microcrystalline cellulose, Croscarmellose Sodium, Magnesium stearate

Table 9 provides illustrative example of a chewable gummy composition containing β-glucogallin and mucic acid gallates for regulating carbohydrate homeostasis

TABLE 9
β-glucogallin Gummy composition

Active Ingredients
β-glucogallin and mucic acid gallates (50-500 mg), Pectin, Glucose corn
syrup
Excipients
Citiric acid, Lactic acid, Lemon peel oil (flavor), DL Tartaric acid,
refinated sugar

The above formulations are just illustrative examples, any formulation containing the above active ingredient intended for the said purpose will be considered equivalent.

Other modifications and variations to the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention.

We claim:

1. A method of inhibiting key enzymes involved in carbohydrate metabolism, said method comprising steps of:
i) Bringing into contact one of the key enzymes in carbohydrate metabolism with a suitable substrate;
ii) Incubating with an effective concentration of a composition containing at least 10% w/w of 1-O-galloyl-β-D-glucose-(β-glucogallin) and 10% w/w to 60% w/w mucic acid gallates under optimal conditions;
iii) Reading the change in absorbance using spectrophotometric and fluorimetric methods
iv) Comparing the absorbance with a control blank and determining the percentage enzyme inhibition (IC₅₀) by the said composition containing at least 10% w/w of 1-O-galloyl-β-D-glucose (β-glucogallin) and 10% w/w to 60% w/w mucic acid gallates using the formula:
% Inhibition=[(absorbance of control−absorbance of inhibitor)/absorbance of control]×100.

2. The method as in claim 1, wherein the mucic acid gallates are selected from the group consisting of mucic acid 1,4-lactone 5-O-gallate, mucic acid 2-O-gallate, mucic acid 6-Methyl ester 2-O-gallate, mucic acid 1-Methyl ester 2-O-gallate and ellagic acid.

3. The method as in claim 1, wherein the enzymes are selected from the list consisting of pancreatic α-amylase, salivary α-amylase, α-glucosidase and dipeptidylpeptidase-4.

4. A composition containing at least 10% w/w of 1-O-galloyl-β-D-glucose (β-glucogallin) and 10% w/w to 60% w/w mucic acid gallates for the management of hyperglycemic conditions in mammals by inhibiting carbohydrate absorption, and normalizing the metabolism of glucose.

5. The composition as in claim 4, wherein the mucic acid gallates are selected from the group consisting of mucic acid 1,4-lactone 5-O-gallate, mucic acid 2-O-gallate, mucic acid 6-Methyl ester 2-O-gallate, mucic acid 1-Methyl ester 2-O-gallate and ellagic acid.

6. The composition as in claim 4, wherein the effective regulation of carbohydrate absorption and breakdown reduces hyperglycemic conditions present in disease states selected from the group consisting of diabetes, obesity, hyperlipoproteinemia, hyperlipidemia, cardiovascular complications, cancer, atherosclerosis, allergy, inflammation, and osteoporosis.

7. The composition as in claim 4, wherein the mammal is human.

8. The composition as in claim 4, wherein the said composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.

9. A method for the therapeutic management of hyperglycemia in mammals by inhibiting the activity of key enzymes, said method comprising steps of administering a composition containing at least 10% w/w of 1-O-galloyl-β-D-glucose (β-glucogallin) and 10% w/w to 60% w/w mucic acid gallates, to bring about the inhibition of key enzymes involved in carbohydrate metabolism.

10. The method as in claim 9, wherein the inhibition of enzymes results in decreasing blood glucose levels by regulating the release of glucose into the blood and cellular uptake of glucose from the blood stream.

11. The method as in claim 9, wherein the mucic acid gallates are selected from the group consisting of mucic acid 1,4-lactone 5-O-gallate, mucic acid 2-O-gallate, mucic acid 6-Methyl ester 2-O-gallate, mucic acid 1-Methyl ester 2-O-gallate and ellagic acid.

12. The method as in claim 9, wherein the enzymes are selected from the list consisting of pancreatic α amylase, salivary α amylase, α glucosidase and dipeptidyl peptidase-4.

13. The method as in claim 9, wherein the hyperglycemic condition is present in disease states selected from the group consisting of diabetes, obesity, hyperlipoproteiniemia, hyperlipidemia, cardiovascular complications, cancer, atherosclerosis, allergy, inflammation, and osteoporosis. In another related embodiment, the mammal is human.

14. The method as in claim 9, wherein the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.

15. A method for the therapeutic management of hyperglycemia by increasing secretion of insulin in mammals, using a composition containing at least 10% w/w of 1-O-galloyl-β-D-glucose (β-glucogallin) and 10% w/w to 60% w/w mucic acid gallates to said mammals, to bring about the inhibition of enzyme dipeptidyl peptidase-4 thereby promoting insulin secretion and increased cellular uptake of glucose from the blood.

16. The method as in claim 15, wherein the mucic acid gallates are selected from the group consisting of mucic acid 1,4-lactone 5-O-gallate, mucic acid 2-O-gallate, mucic acid 6-Methyl ester 2-O-gallate, mucic acid 1-Methyl ester 2-O-gallate and ellagic acid.

17. The method as in claim 15, wherein the said composition is used for the management of hyperglycemia present in disease conditions selected from the group consisting of diabetes, obesity, hyperlipoproteinemia, hyperlipidemia, cardiovascular complications, cancer, atherosclerosis, allergy, inflammation, and osteoporosis.

18. The method as in claim 15, wherein the mammal is human.

19. The method as in claim 15, wherein the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.
